(19) United States
(12) Patent Application Publication
(10) Pub. No.: US 2022/0086572 A1
(43) Pub. Date: Mar. 17, 2022

(54) CONTACT HEARING PROTECTION DEVICE

(71) Applicant: Earlens Corporation, Menlo Park, CA (US)

(72) Inventors: Bryan FLAHERTY, Mountain View, CA (US); Jeffrey JONES, San Mateo, CA (US); Morteza KHALEGHIMEYBODI, Bothell, WA (US); Paul RUCKER, San Francisco, CA (US)

(21) Appl. No.: 17/532,949

(22) Filed: Nov. 22, 2021

Related U.S. Application Data
(63) Continuation of application No. 16/790,455, filed on Feb. 13, 2020.
Application No. 16/790,455 is a continuation of application No. PCT/US18/49945, filed on Sep. 7, 2018.
(60) Provisional application No. 62/558,224, filed on Sep. 13, 2017.

(51) Int. Cl. H04R 25/00

(52) U.S. Cl. CPC H04R 25/30; H04R 25/652; H04R 2460/13; H04R 2225/61; H04R 2225/41

(57) ABSTRACT

A contact hearing protection device is provided. The contact hearing protection device comprises a perimeter platform, a chassis connected to the perimeter platform, a control system including at least one variable limiting element, and an umbo platform connected to the at least one limiting element. A sound level which meets or exceeds a predetermined level is detected, as can be indicated by an increased magnitude of movement of the umbo platform, and the limiting element increases the limiting in response to the increased magnitude of movement of the umbo platform.

### CROSS REFERENCE TO RELATED APPLICATIONS

This application is a continuation of U.S. patent application Ser. No. 16/790,455, filed Feb. 13, 2020; which is a continuation of PCT Application No. PCT/US18/49945, filed Sep. 7, 2018; which claims the benefit of U.S. Provisional Application No. 62/558,224, filed Sep. 13, 2017; which are incorporated herein by reference in their entirety.

### BACKGROUND OF THE INVENTION

### Field of the Invention

The present invention is directed to hearing systems and, more, particularly, to hearing systems which may be used to protect a wearer's hearing from sounds which might damage elements of the user's auditory system.

### BACKGROUND

Loud sounds may be damaging to components of the human ear. Sounds create a pressure wave in the ear canal which vibrates the eardrum, which, in turn vibrates other elements of the auditory system, resulting in the perception of sound. At times, such vibrations reach a magnitude or duration which results in damage to elements of the auditory system. In particular, both the eardrum and elements of the cochlea are susceptible to such damage. dB SPL is an abbreviation of decibel sound pressure level, it is a measurement of sound pressure, expressed in decibels, with respect to the threshold of hearing of a particular patient. The threshold of hearing is usually defined as 20 micro-pascals, which may be assigned a value of 0 dB SPL. Leaves gently rustling produce a sound level of approximately 15 dB SPL, a whisper is about 30 dB SPL, the dial tone of a telephone is approximately 80 dB SPL and an approaching subway train may reach approximately 110 dB SPL. Naturally, each of these approximations may vary quite a bit. For instance, the type of train, its approach speed and station acoustics are some of the factors which affect a dB SPL reading in the case of a subway. For a person with good hearing, pain begins somewhere around 120 dB SPL, and there is immediate damage to hearing above 150 dB SPL (some studies have shown that the eardrum may be damaged when sound levels exceed 160 dB SPL). Further, the frequency content of the sound waves may have detrimental effects on the hearing system of a listener. In particular, loud sounds at high frequencies within the audible range may cause more damage than lower frequency sounds having the same decibel level. Movement at lower frequencies (e.g., 100 to 400 Hz) have less energy, making them less likely to damage the components of the hearing system, while movement at higher frequencies (e.g., 5 KHz to 10 KHz) have much greater energy, making it more likely that inducing vibrations at those, higher, frequencies will cause damage to components of the hearing system. It would, therefore, be advantageous to design a hearing protection system which provide hearing protection at the eardrum of the listener.

### SUMMARY OF THE INVENTION

The present invention includes a contact hearing protection device and methods for protecting the auditory system. The present invention is adapted to protect the listener from both impulse sounds and from loud continuous sounds. The present invention incudes devices and methods for protecting the hearing of a listener through mechanisms positioned in contact with the eardrum of the user.

A contact hearing protection device according to the present invention is designed to allow a listener to hear sounds below a predetermined level, e.g., a level which would cause damage to the listener's auditory system, while limiting the magnitude and/or duration of vibration of the listener's eardrum when sound pressure levels reach or exceed the predetermined level, in order to prevent damage to the listener's auditory system. In embodiments of the invention, the hearing protection device is designed to limit the magnitude and/or duration of movement of the eardrum without completely preventing the eardrum from moving, thereby allowing sounds through even when protecting the eardrum from excessive noise or excessive sound pressure. As used herein, the term “limiting” may also refer to damping such as, for example, damping vibrations of the eardrum. Embodiments of the invention may further include components which actively amplify and transmit the external sound in order to assist listeners with hearing loss, while also including components which protect the listener's hearing when sound pressure levels reach or exceed the predetermined damaging levels.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a side view of a contact hearing protection device according to the present invention.

FIG. 1A is a side view of a contact hearing protection device according to the present invention wherein the contact hearing protection device includes a membrane.

FIG. 1B is a side view of a contact hearing protection device according to the present invention, the contact hearing device being positioned in the ear canal of a user, wherein the contact hearing device includes an umbrella structure.

FIG. 1C is a top perspective view of a contact hearing protection device according to the present invention wherein the contact hearing device includes an umbrella structure.

FIG. 1D is a top view of a contact hearing protection device according to the present invention wherein the contact hearing device includes an umbrella structure.

FIG. 1E is a bottom view of a contact hearing protection device according to the present invention wherein the contact hearing device includes an umbrella structure.

FIG. 2 is a side view of a contact hearing protection device according to the present invention, wherein the contact hearing protection device includes a latching mechanism.

FIG. 2A is a further side view of the contact hearing protection device of FIG. 2.

FIG. 3 is a side view of a contact hearing protection device according to the present invention where the contact hearing protection device is positioned in the ear canal of a user.

FIG. 3A is a side view of an alternative contact hearing protection device according to the present invention where the contact hearing protection device is positioned in the ear canal of a user.

FIG. 4 is a side view of a contact hearing protection device according to the present invention where contact hearing protection device is positioned in the ear canal of a user.

FIG. 4A is a side view of an alternative contact hearing protection device according to the present invention where contact hearing protection device is positioned in the ear canal of a user.

FIG. 5 is a top view of a membrane structure according to the present invention.

FIG. 6 is a schematic diagram of a control system for a contact hearing device according to the present invention.

FIG. 6A is a schematic diagram of a control system for a contact hearing device according to the present invention.

FIG. 6B is a schematic diagram of a control system for a contact hearing device according to the present invention.

FIG. 7 is a graph showing a force verses displacement profile for a control system for a contact hearing device according to the present invention.

FIG. 7A is a graph showing a stiffness verses displacement profile for a control system for a contact hearing device according to the present invention.

FIG. 8 is a schematic diagram of a control system for a contact hearing device according to the present invention.

FIG. 9 is a schematic diagram of a control system for a contact hearing device according to the present invention.

FIG. 10 is a schematic diagram of a control system for a contact hearing device according to the present invention.

FIG. 11 is a schematic diagram of a control system for a contact hearing device according to the present invention.

FIG. 12 is a schematic diagram of a control system for a contact hearing device according to the present invention.

### DETAILED DESCRIPTION OF THE INVENTION

### Apparatus Detail

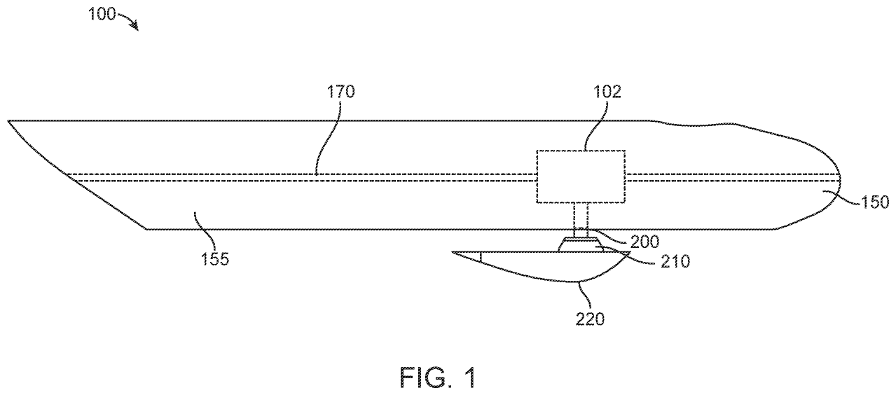

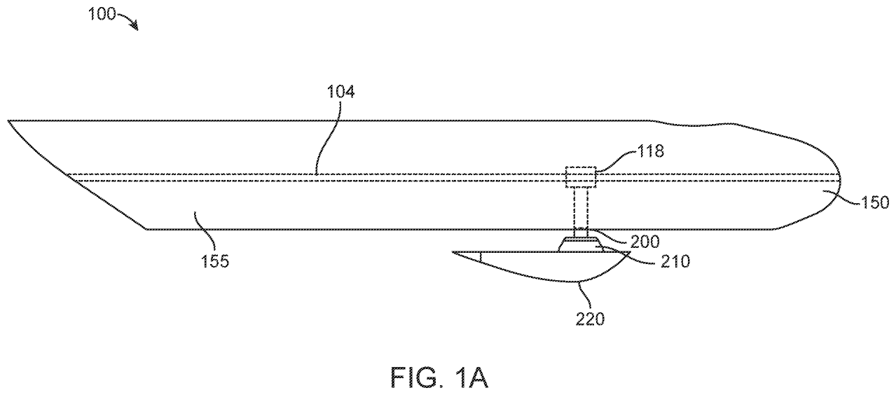

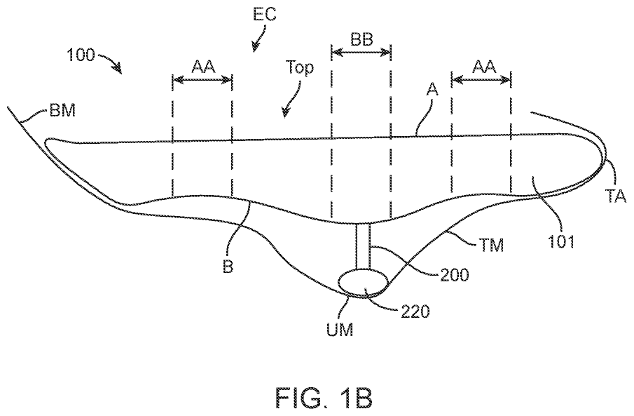

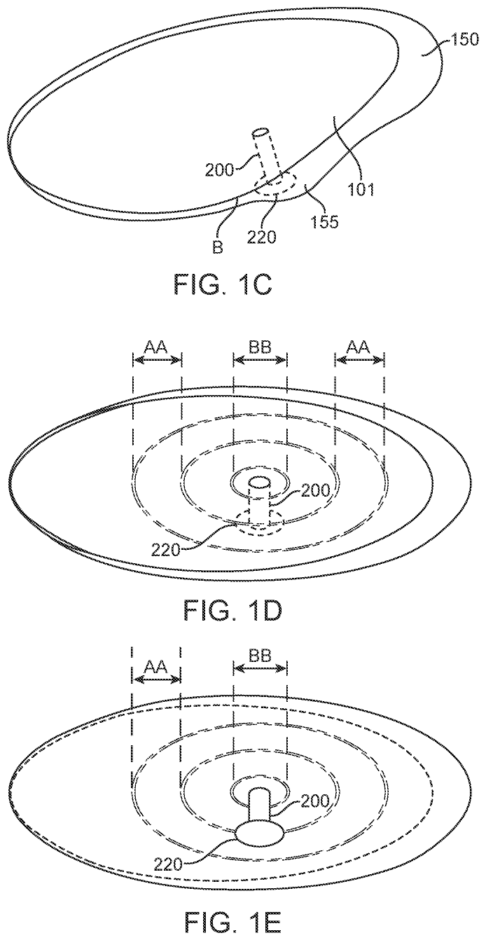

FIG. 1 illustrates a contact hearing protection device 100 according to the present invention, wherein the contact hearing protection device includes a control mechanism 102. The contact hearing device further includes a perimeter platform 155 which incorporates a sulcus platform 150. A chassis 170 is attached to perimeter platform 155 and supports control mechanism 102. Drive post 200 extends from control mechanism 102 and is attached to umbo lens 220 by adhesive 210.

FIG. 1A illustrates a contact hearing protection device 100 according to the present invention, wherein contact hearing protection device 100 includes membrane 104. Contact hearing device 100 further includes perimeter platform 155 which incorporates sulcus platform 150, drive post 200 and umbo lens 220. Membrane 104 may be connected to drive post 200 by connector 118. Drive post 200 is connected to umbo lens 220 by adhesive 210.

FIG. 1B is a side view of a contact hearing protection device 100 according to the present invention wherein the contact hearing device includes an umbrella structure. FIG. 1C is a top perspective view of a contact hearing protection device according to the present invention wherein the contact hearing device includes an umbrella structure. FIG. 1D is a top view of a contact hearing protection device according to the present invention wherein the contact hearing device includes an umbrella structure. FIG. 1E is a bottom view of a contact hearing protection device according to the present invention wherein the contact hearing device includes an umbrella structure. While embodiments of the invention attenuate very loud sounds using mechanical elements such as non-linear springs and viscous mechanisms, an umbrella-type structure can also protect an eardrum from being ruptured, perforated or damaged in the presence of very loud sounds. As illustrated in FIG. 1B, the umbrella structure will not be in contact with the main portion of the eardrum (which may also be referred to as the Tympanic Membrane or TM), sitting above the eardrum and being held in place by perimeter platform 155. The shape of the portion of umbrella structure 101 which is positioned above and separated from the protected eardrum, the protective membrane 103, may be adapted to follow the geometry of the protected eardrum as shown in FIG. 1B. In embodiments of the invention, the region of protective membrane 103 which is designed to be positioned over the umbo region of the protected eardrum, the umbo region BB, may be designed to have a concave cross section such that the interior portion of umbo region BB is extends toward the protected eardrum when umbrella structure 101 is positioned next to the protected eardrum. In embodiments of the invention the region of protective membrane 103 which surrounds umbo region BB, the annular region AA, may be designed to have a convex cross section with respect to umbo region BB, such that a central portion of annular region AA extends away from the protected eardrum when umbrella structure 101 is positioned next to the protected eardrum. In FIG. 1B, the portion of protective membrane 103 which is positioned over the umbo of the protected eardrum may be attached to drive post 200. This non-linear umbrella structure will be relatively transparent in low-magnitude sound pressure levels, however, as the sound pressure level goes higher, the equivalent stiffness and damping characteristics of this umbrella structure also increases, non-linearly. Thus, this mechanism will prevent very loud sounds from reaching the eardrum while still letting normal-level sounds through to the protected eardrum.

In the embodiment of the invention illustrated in FIGS. 1B-1E, sound pressure incident on protective membrane 103 will cause protective membrane 103 to vibrate, which will, in turn result in the movement of drive post 200 and umbo lens 220. When umbo lens 220 is in contact with the protected eardrum, those vibrations will be transmitted through to the protected eardrum. Should the sound pressure incident on protective membrane 103 exceed a predetermined pressure, the interaction of umbo region BB and annular region AA will result in an increased stiffness which opposes the motion induced by the incident sound pressure (sound waves) and the umbo lens will not move in proportion to the increase in sound pressure. Thus preventing damage to the eardrum and/or other components of the hearing system.

For example, with low level sounds the vibration will cause the eardrum to vibrate, which, in turn, will result in movement of umbo lens 220. When the magnitude of the movements are small, the umbrella structure will provide very little resistance to those movements since the umbo region BB is free to move. When the magnitude of the movements reached a predetermined level, umbo region BB will go from being concave and extending towards the eardrum to being convex and extending away from the eardrum. When the umbo region BB reaches the limit of its movement away from the eardrum it will present an substantial barrier to further movement of the eardrum through umbo lens 220, limiting the magnitude of that movement and thereby, damage done to the eardrum and/or other components of the user's hearing system.

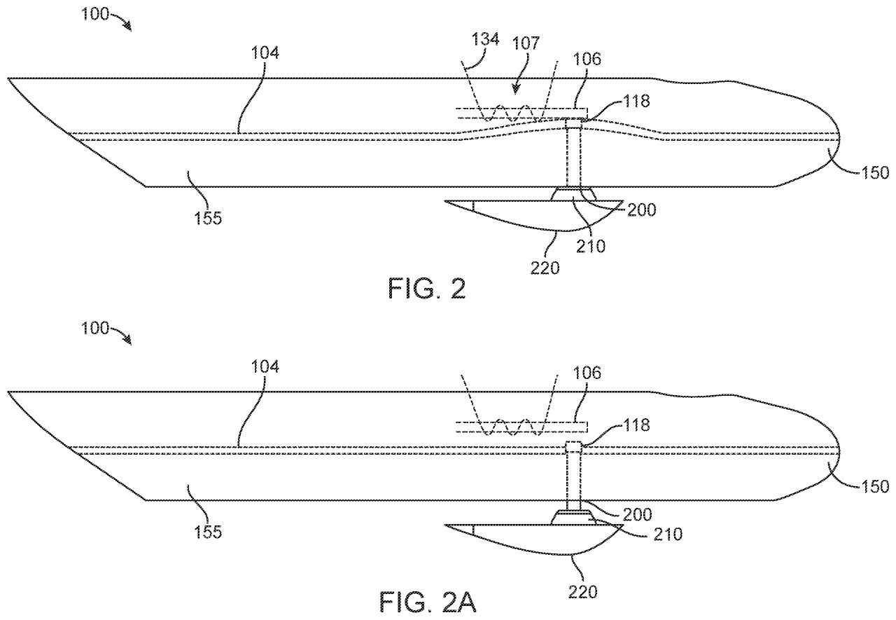

FIGS. 2 and 2A illustrate a contact hearing protection device 100 according to the present invention, wherein contact hearing protection device 100 includes a magnetic latching mechanism 106. Contact hearing device 100 further includes perimeter platform 155 which incorporates sulcus platform 150, drive post 200 and umbo lens 220. Membrane 104 is connected to drive post 200 by connector 118. Drive post 200 is connected to umbo lens 220 by adhesive 210. Magnetic latching mechanism 106 may, in some embodiments, comprise an electromagnet 107 including a drive coil 134. Connector 118 may, in some embodiments, include a magnet or ferrous material. In operation, contact hearing protection device 100 may be positioned in the ear canal of a user. When in that position, magnetic latching mechanism 106 may hold umbo lens 220 away from the user's eardrum so that sound pressure reaching the eardrum would cause the eardrum to vibrate normally, transmitting that sound to the user. However, once the sound pressure in the user's ear canal reaches a predetermined level, e.g., a level which could result in damage to the user's hearing, latching mechanism 106 may be released by, for example, halting or reversing current flowing through drive coil 134, which would allow umbo lens to move into contact with the user's eardrum through, for example, forces applied by membrane 104. Once umbo lens 220 is in contact with the user's eardrum, movement of the umbo lens and the eardrum will be restricted by the force exerted by membrane 104, limiting the magnitude of the movement induced by the incident sound pressure. Once the incident sound pressure is reduced to an acceptable level, electromagnet 107 may be reactivated, by, for example, putting current through drive coil 134, which pulls umbo lens 220 away from the user's eardrum and allows the eardrum to vibrate freely.

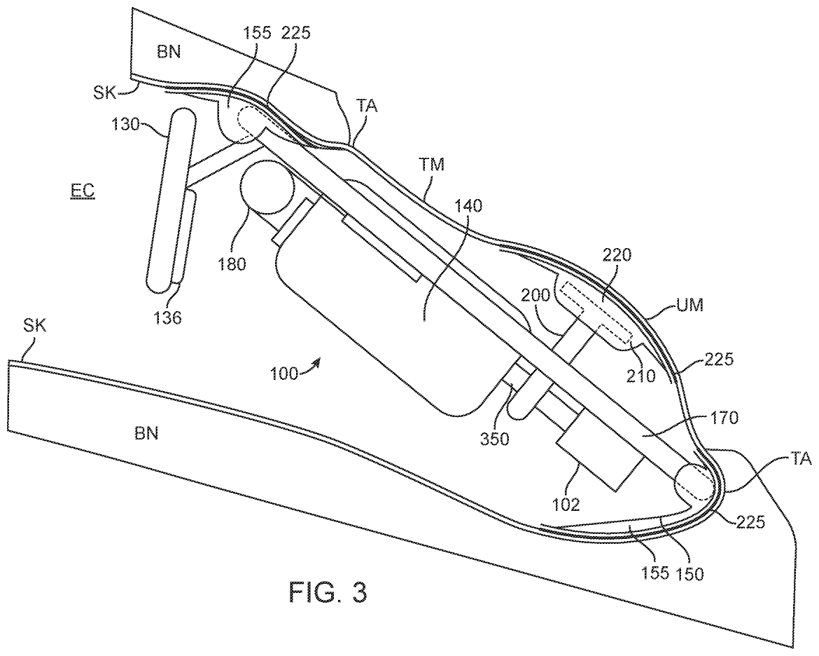

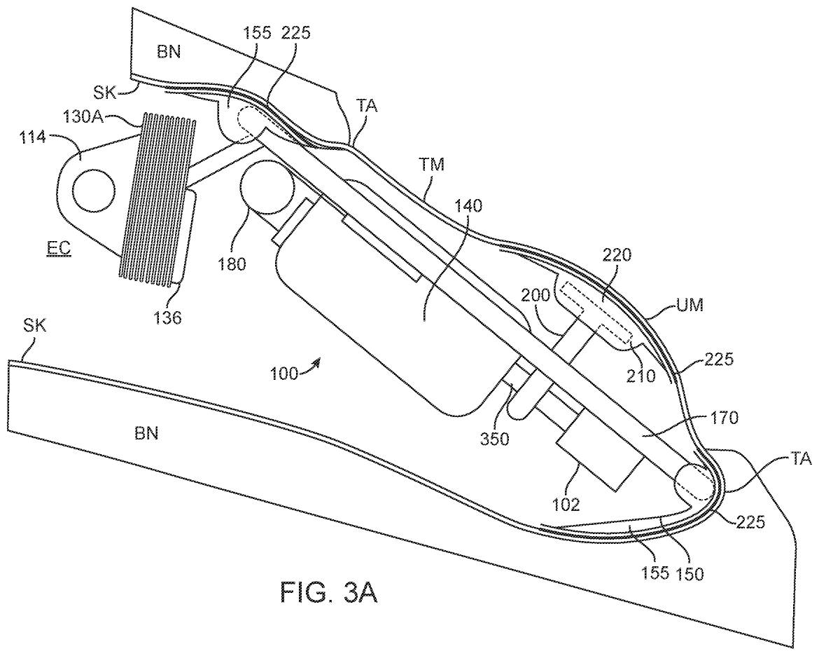

FIG. 3 is a side view of a contact hearing protection device 100 according to the present invention where contact hearing protection device 100 is positioned in the ear canal EC and in contact with the eardrum TM of a listener. In FIG. 3, contact hearing device 100 includes perimeter platform 155 which includes sulcus platform 150 at a distal end thereof. Perimeter platform 155 is connected to chassis 170, which supports microactuator 140 through bias springs 180, which may also be referred to as torsion springs. Microactuator 140 includes microactuator reed 350 extending from a distal end thereof. Microactuator reed 350 is connected to umbo lens 220 through drive post 200. Chassis 170 further supports photodetector 130, which is electrically connected to microactuator 140. In embodiments of the invention, photodetector 130 may be replaced by, for example, an inductive coil or RF antenna. In FIG. 3, perimeter platform 155 is positioned on skin SK covering the boney portion BN of the ear canal EC. The sulcus platform 150 portion of perimeter platform 155 is positioned at the medial end of the ear canal in the tympanic annulus TA. Umbo lens 220 is positioned on umbo UM of eardrum UM. In FIG. 3, microactuator reed 350 extends into control mechanism 102. In embodiments of the invention, an oil layer 225 may be positioned between the skin SK and the perimeter platform 155. In embodiments of the invention, contact hearing protection device 100 may further include an electronics package 136. FIG. 3A is a side view of an alternative contact hearing protection device 100 according to the present invention where the contact hearing protection device 100 is positioned in the ear canal of a user. In FIG. 3A photodetector 130 may be replaced by receive coil 130A which may be useful in a system wherein information, including control information, and/or power are transmitted to contact hearing device 100 thorough inductive coupling. The embodiment of FIG. 3A may further include grasping tab 114A.

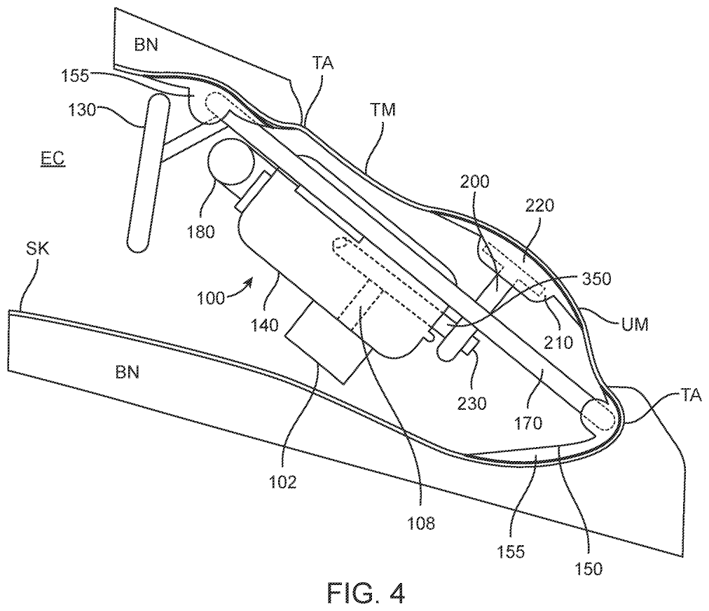

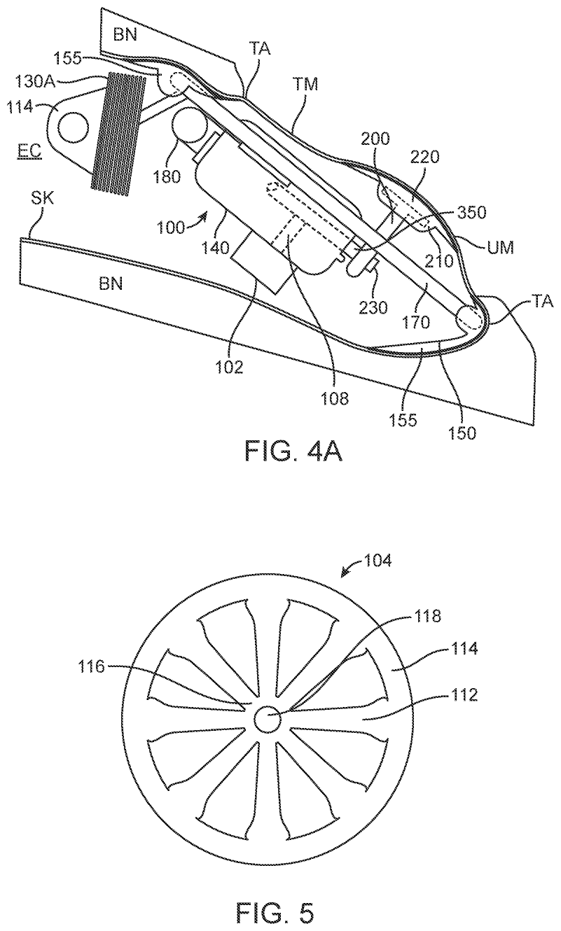

FIG. 4 is a side view of a contact hearing protection device 100 according to the present invention where contact hearing protection device 100 is positioned in the ear canal EC and in contact with the eardrum TM of a listener. In FIG. 4, contact hearing device 100 includes perimeter platform 155 which includes sulcus platform 150 at a distal end thereof. Perimeter platform 155 is connected to chassis 170, which supports microactuator 140 through bias springs 180. Microactuator 140 includes microactuator reed 350 extending from a distal end thereof. Microactuator reed 350 is connected to umbo lens 220 through drive post 200. Chassis 170 further supports photodetector 130, which is electrically connected to microactuator 140. In FIG. 4, perimeter platform 155 is positioned on skin SK covering the boney portion BN of the ear canal EC. The sulcus platform 150 portion of perimeter platform 155 is positioned at the medial end of the ear canal in the tympanic annulus TA. Umbo lens 200 is positioned on umbo UM of eardrum TM. In FIG. 3, microactuator reed 350 is connected to control mechanism 102 through control shaft 108. In embodiments of the invention oil layer 225 may be positioned between skin SK and perimeter platform 155. FIG. 4A is a side view of an alternative contact hearing protection device 100 according to the present invention where contact hearing protection device 100 is positioned in the ear canal of a user. In FIG. 4A photodetector 130 of FIG. 4 may be replaced by receive coil 130A which may be useful in a system wherein information and power are transmitted to contact hearing device 100 thorough inductive coupling. The embodiment of FIG. 4A may further include grasping tab 114A.

In the embodiments of FIGS. 3, 3A, 4 and 4A, components of the system may act as a contact hearing aid, providing the user with an enhanced audio signal to enable the user to hear sounds that would not be audible to the user without the aid of the hearing aid, while components of the system may act as a contact hearing protection device, protecting the user's hearing when external stimuli, e.g. sound pressure, results in levels that may be damaging to the hearing of the listener. In these systems, control mechanism 102 may act in concert with umbo lens 220, drive post 200 and microactuator reed 350 to form at least a part of a contact hearing device according to the present invention. Methods and structures for implementing control mechanism 102 according to the present invention will be described herein.

FIG. 5 illustrates a membrane 104 according to the present invention. Membrane 104 includes perimeter 114, radial supports 112, and connector ring 116. Connector ring 116 includes connector 118 comprising an opening adapted to receive and hold drive post 200. In embodiments of the invention, such as the embodiment illustrated in FIG. 2, membrane 104 may be positioned on contact hearing protection device 100 by, for example, connecting membrane 104 to chassis 170. Umbo lens 220 may be connected to membrane 104 by affixing drive post 200 to connector 118 in connector ring 116.

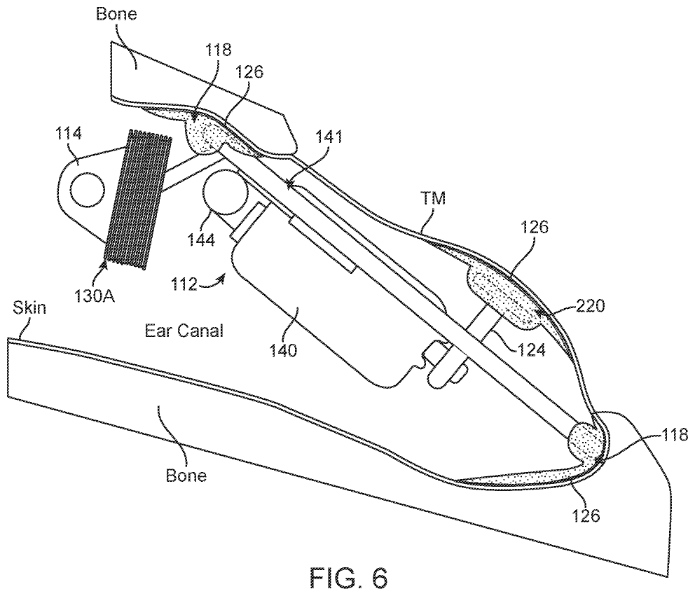

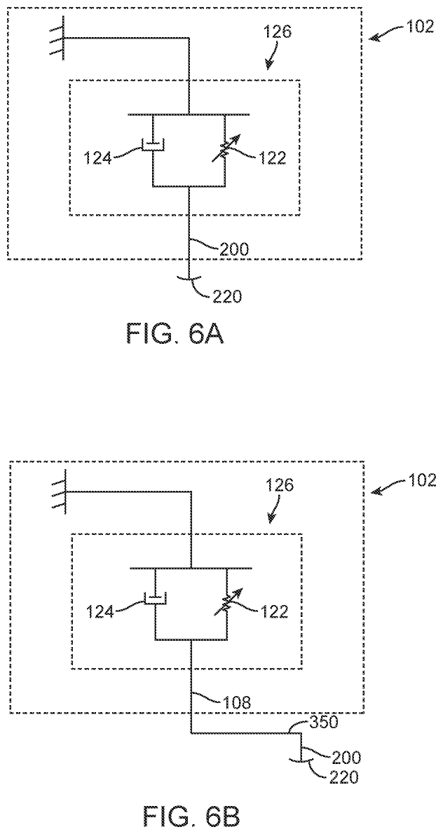

FIG. 6 is a schematic diagram of a control system 126 for use in control mechanism 102 in a contact hearing device 100 according to the present invention. In FIG. 6, umbo lens 220 is connected to control mechanism 102 by drive post 200. In control mechanism 102, drive post 200 is connected to control system 126. Control system 126 may include non-linear spring element 122 and damper 124. In embodiments of the invention, suitable non-linear springs may include non-linear springs having characteristics which may be used to implement systems which operate according to the Duffing Equation. In embodiments of the invention, damper 124 may be a viscous element. In embodiments of the invention, damper 124 may be a constant viscous element (that is an element where the resistance to movement is proportional to the velocity of that movement) or a non-linear viscous element (that is an element where the resistance to movement it non-linear with respect to the velocity of that movement). In embodiments of the invention, control system 126 acts as a mechanical interconnection between umbo lens 220 and perimeter platform 155, wherein control system 126 is designed to limit the amplitude of movement of umbo lens 220, and thus the amplitude of movement of the user's eardrum across a range of frequencies. The mechanical interconnection may limit the amplitude of movement in a non-linear fashion (e.g., allowing more movement at lower sound pressure levels and limiting movement at higher sound pressure levels). The mechanical interconnection may further limit the amplitude of movement differently across a range of frequencies (e.g., the amplitude of movement may be more restricted at higher frequencies than at lower frequencies).

FIG. 6A is a schematic diagram of a control system 126 for use in control mechanism 102 in a contact hearing device 100 according to the present invention. In FIG. 6A, umbo lens 220 is connected to controls system 126 (which may include damper 124 and spring element 122) by drive post 200.

FIG. 6B is a schematic diagram of a control system 126 for use in control mechanism 102 in a contact hearing device 100 according to the present invention. In FIG. 6B, umbo lens 220 is connected to microactuator reed 350 by drive post 200 and microactuator reed 350 is connected to control mechanism 102 by control shaft 108. In control mechanism 102, drive post 200 is connected to control system 126. Control system 126 may include non-linear spring element 122 and damper 124.

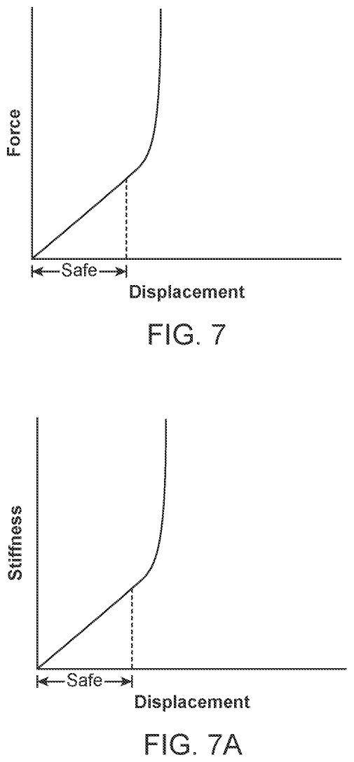

FIG. 7 is a graph showing an optimal force verses displacement profile for a control system 126 according to the present invention. In FIG. 7, when the displacement of, for example, the eardrum, is within a safe region, the force applied by control system is proportional to the movement of the eardrum, resulting in a substantially linear relationship between force and displacement. However, when the displacement of the eardrum exceeds a predetermined minimum, wherein movement exceeding the predetermined minimum is sufficient to cause damage, the force is increased in a non-linear fashion to limit displacement and, therefore, damage.

FIG. 7A is a graph showing an optimal stiffness verses displacement profile for a control system 126 according to the present invention. In FIG. 7A, when the displacement of, for example, the eardrum, is within a safe region, the stiffness (e.g. it's resistance to motion) of control system 126 is minimized, resulting in very little resistance to movement being applied to the eardrum. However, when the displacement of the eardrum is sufficient to cause damage (outside the “safe region”), the stiffness of the control system is increased in a non-linear fashion to limit displacement of the eardrum and, therefore, damage to the eardrum is prevented or minimized.

FIG. 8 is a diagram of a control mechanism 102 for a contact hearing device 100 according to the present invention. In FIG. 8, control system 126 includes membrane 104 which is connected to umbo lens 220 by drive post 200. Membrane 104 in FIG. 8 is pre-stressed to include one or more concave 132 (with respect to drive post 200) and convex 128 sections. The position and number of concave 132 and convex 128 sections are chosen to provide a predetermined force-displacement profile for control system 126. In embodiments of the invention, small movements of umbo lens 220 will result in movements of membrane 104 which are relatively unconstrained, however, once movements of umbo lens 220 force membrane 104 to fully stretch out, the further movement of umbo lens 220 will be restricted and/or prevented all together.

FIG. 9 is a schematic diagram of a control system for a contact hearing device according to the present invention. In FIG. 9, umbo lens 220 is connected to drive post 200, which is connected to flexible support 111. The range of motion of flexible support 111 is limited by travel stops 110. In embodiments of the invention, flexible support 111 and travel stops 110 may be positioned on, for example chassis 170 of a contact hearing device 100. In operation, with umbo lens 220 in contact with the eardrum of a user, flexible support 111 provides for limited relatively free movement of umbo lens 220 over a small range of motion. However, when the range of motion exceeds a predetermined limit, bringing flexible support 111 into contact with travel stop 110, the resistance to continued motion increases substantially, depending upon the flexibility of the distal end of flexible support 111. Thus, motion transmitted from the eardrum through umbo lens 220 and drive post 200 to travel stop 110 is limited in magnitude by the presence of travel stops 110. In embodiments of the invention, the control system illustrated in FIG. 9 may further incorporate magnetic stops such as those shown in FIG. 11.

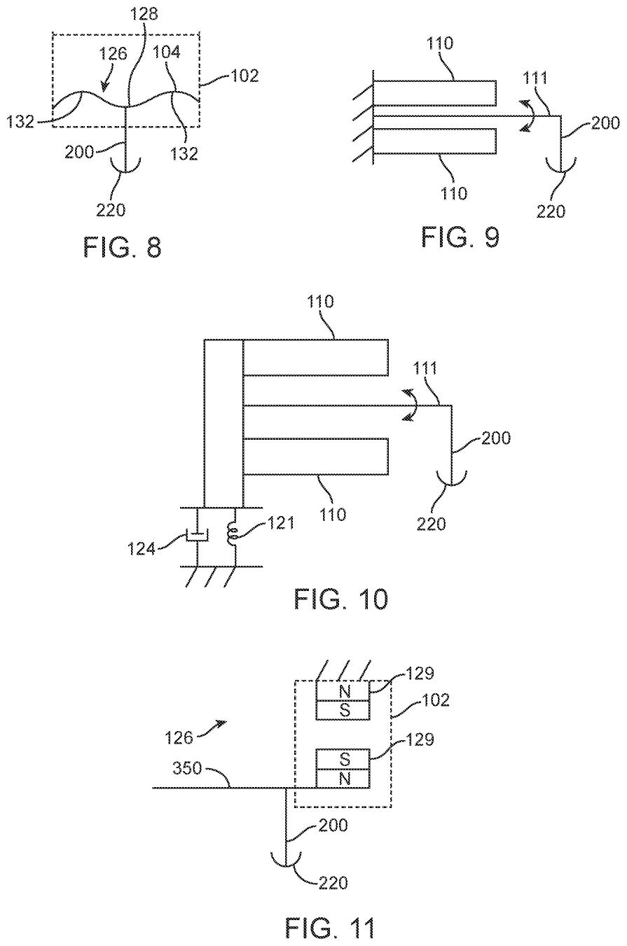

FIG. 10 is a schematic diagram of a control system for a contact hearing device according to the present invention. In FIG. 10, the system of FIG. 9 may be connected to, for example, chassis 170 by an energy absorbing element including, for example, spring 121 and damper 124. In embodiments of the invention, spring 121 may be a non-linear spring such as non-linear spring 122 described herein.

FIG. 11 is a schematic diagram of a control system for a contact hearing device according to the present invention. In the embodiment of FIG. 11, control mechanism 102 may include permanent magnets 129, which magnets may be positioned such that motion of umbo lens 220, transmitted through drive post 200 to, for example, microactuator reed 350 brings the like poles of permanent magnets 129 toward each other. As the magnitude of the movement of umbo lens 220 increases, the distance between the like poles decreases, thereby increasing the force opposing movement of the umbo lens 220 in a non-linear manner until the movement is stopped all together by contact between permanent magnets 129. In embodiments of the invention, the repulsive force between the magnets is a function of the square of the distance between the like poles.

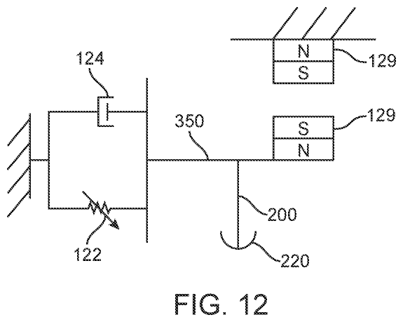

FIG. 12 is a schematic diagram of a control system for a contact hearing device according to the present invention. In FIG. 12, the system of FIG. 11 may be connected to, for example, chassis 170 by an energy absorbing element including, for example, damper 124 and non-linear spring 122.

### Function

In one embodiment of the invention, control mechanism 102 may include either active or passive control mechanisms or circuitry. The mechanisms and circuitry in control mechanism 102 are designed to dampen the vibration of the eardrum (not shown in FIG. 1) when sound pressure in the ear canal meets or exceeds a damage threshold. Vibrations in the eardrum may be dampened through the interaction of umbo lens 220 with the eardrum. In embodiments of the invention, umbo lens 220 may be continuously in contact with the eardrum or it may be brought into contact upon the detection of sound pressure which meets or exceeds a predetermined threshold such as a damage threshold.

For a system where umbo lens 220 is in continuous contact with the eardrum, the resistance of umbo lens 220 and the control mechanisms (e.g., control system 126) attached to umbo lens 220 to vibrations in the eardrum may be calibrated to the sound pressure level reaching the listener. For example, when the sound pressure level is below a damage threshold, umbo lens 220 may be pulled away from or present little or no resistance to the movement of the eardrum of the listener. Thus, when the sound pressure level is below the damage threshold, the eardrum vibrates freely. Alternatively, when the sound pressure level reaches or exceeds the damage threshold, umbo lens 220 may present significant resistance to the vibration of the eardrum, preventing damage to the auditory system of the user. The resistance of umbo lens 220 to movement of the eardrum may therefore, be correlated to the sound level perceived by the listener, with greater resistance above a damage threshold and lesser or no resistance below the damage threshold. In embodiments of the invention, the stiffness of the control system attached to umbo lens 220 may be correlated to the sound level perceived by the listener, with greater stiffness above a damage threshold and lesser or no stiffness below the damage threshold.

For a system where umbo lens 220 is not in continuous contact with the eardrum of the user, it may be brought into contact when a sound level meeting or exceeding a damage threshold is detected. Once the damage threshold is detected, the umbo lens 220 may be placed against the eardrum to provide resistance to unwanted vibrations. Umbo lens 220 could thereafter be lifted off the eardrum when the sound level drops below the damage threshold, allowing the listener to hear sounds without interference.

In embodiments of the invention, umbo lens 220 may be a component of a contact hearing aid wherein the umbo lens is adapted to vibrate the eardrum in response to stimulus from an external source, such as a hearing aid signal processor (e.g., a BTE). In these systems, for sound levels below he damage threshold, the umbo lens would be used to amplify incoming sounds in order to enhance the user's hearing, however, for sound levels at or above the damage threshold, umbo lens 220 would be used to dampen the vibration of the tympanic lens, protecting the auditory system of the listener.

In embodiments of the invention, sound levels which meet or exceed the damage threshold may be detected either actively or passively. In an active detection system contact hearing protection device 100 may be activated (e.g., the resistance or engagement of umbo lens 220 may be initiated) by a signal from an external source, such as, for example, a sound processor, which detects a sound at or above the damage threshold and engages mechanisms, including umbo lens 220 which dampen the motion of the eardrum until the external sound drops below the damage threshold.

In a passive detection system, contact hearing protection device 100 may be activated by, for example, the amplitude of vibrations in the eardrum. In passive systems, the amplitude of vibration may, for example, trigger limiting mechanisms in control mechanism 102, which limiting mechanisms dampen the vibration of the eardrum while the sound level meets or exceeds the damage threshold.

In one embodiment of the invention, illustrated in FIG. 1A, umbo platform 220 may be mounted on a membrane 104, such that, when umbo lens 220 is in contact with the eardrum of a listener, vibrations of the eardrum are transmitted through umbo lens 220 to membrane 104. In one embodiment of the invention, the viscoelastic characteristics of membrane 104 may be such that, below the damage threshold, the membrane provides little or no resistance to the movement of umbo lens 220 and, at or above he damage threshold, the membrane stiffens to provide increased resistance to movement of umbo lens 220, thus limiting the movement of the tympanic lens.

In one embodiment of the invention, membrane 104 may be the membrane illustrated in FIG. 5 and may have the displacement vs. resistance characteristics illustrated in FIG. 7 and/or FIG. 7A. In FIG. 7, the resistance of membrane 104 increases substantially linearly when the displacement of umbo lens 220 is within the “safe” zone (e.g., the sound level is below the damage threshold) and increases exponentially when the displacement is outside the “safe” zone (e.g., the sound level is above the damage threshold). In FIG. 7A, the resistance of membraned 104 is close to zero when the displacement of umbo lens 220 is within the “safe” zone (e.g., the sound level is below the damage threshold) and increases exponentially when the displacement is outside the “safe” zone (e.g., the sound level is above the damage threshold).

In an embodiment of the invention such as the one illustrated in FIG. 2, a contact hearing protection device 100 may be positioned in an ear canal such that umbo lens 220, which is held in place by magnetic latching mechanism 106 is positioned adjacent the listener's eardrum but does not touch the eardrum. As illustrated in FIG. 2A, when a sound level is detected which meets or exceeds the damage threshold, magnetic latching mechanism may release connector 118 allowing umbo lens 220 to drop down and contact the listener's eardrum, limiting the vibration of the eardrum. The viscoelastic characteristics of membrane 104 may be configured to define the amount of limiting since drive post 200 is connected to membrane 104 through connector 118. Once the sound level drops below the damage threshold, umbo lens 220 may be pulled away from the listener's eardrum by magnetic latching mechanism, removing the limiting and allowing the listener to hear without impediment.

In the embodiment of the invention illustrated in FIG. 3, a contact hearing protection device 100 may include the components of a contact hearing aid, such as microactuator 140 and photodetector 130, which may be combined with a control mechanism 102. In this embodiment, control mechanism 102, acting through umbo lens 220, dampens the vibration of eardrum TM when the sound level meets or exceeds a damage threshold. In the embodiment of FIG. 3, microactuator reed 350, which is connected to umbo lens 220 through drive post 200, extends into control mechanism 102. Control mechanism 102 may include circuitry or mechanisms to dampen the vibration of microactuator reed 350 and, through umbo lens 220, the eardrum when the sound level meets or exceeds a damage threshold, while allowing substantially free motion of microactuator reed 350 when the sound level is below the damage threshold.

In embodiments of the invention, control mechanism 102 may include a control system 126 such as the limiting system illustrated in FIG. 12. In the control system of FIG. 12, a permanent magnet 128 is affixed to microactuator reed 350 and a second permanent magnet 128 is mounted opposite the permanent magnet affixed to microactuator reed 350. In operation, vibration of microactuator reed 350 moves the two permanent magnets toward each other. In this embodiment, the poles of the permanent magnet are arranged such that the force exerted acts to push the magnets apart as they approach each other as a result of the vibratory motion of microactuator reed 350. In permanent magnets the force exerted falls of falls off inversely with the square of the distance between the magnets. Thus, in the illustrated embodiments, when the vibrations of microactuator reed are small, such as when the sound levels are below a damage threshold, the interaction of the magnets is minimized and microactuator reed 350 is allowed to move freely. However, when the vibrations of microactuator reed 350 are larger, such as when the motions of microactuator reed 350 are driven by vibrations of the eardrum resulting from sound levels that meet or exceed a damage threshold, the interaction of the permanent magnets will result in limiting those vibrations, thus protecting the eardrum.

In the embodiment of the invention illustrated in FIG. 4, a contact hearing device 100 may include the components of a contact hearing devices, such as microactuator 140 and photodetector 130, which may be combined with a control mechanism 102 which, acting through umbo lens 220, dampens the vibration of eardrum TM when the sound level meets or exceeds a damage threshold. In the embodiment of the invention illustrated in FIG. 4, control mechanism 102 is connected to microactuator reed 350 by control shaft 108. Control mechanism 102 may include circuitry or mechanisms to dampen the vibration of microactuator reed 350 and the eardrum when the sound level meets or exceeds a damage threshold while allowing substantially free motion of microactuator reed 350 when the sound level is below the damage threshold.

In embodiments of the invention, control mechanism 102 may include a control system 126 such as the control system illustrated in FIG. 6A. The control system 126 in FIG. 6A includes a damper 124 and a non-linear spring 122 which may be adapted to have resistance vs displacement characteristics similar to those illustrated in FIG. 7A, wherein control system 126 provides little or no resistance to the movement of microactuator reed 350 when the vibration is within a safe range but increases the resistance when the displacement exceeds the safe range. Thus, while the sound levels are below a damage threshold, control system 126 exerts little or no force on microactuator reed 350, allowing it to vibrate freely. However, when the displacement of microactuator reed 350 exceeds the safe range, for example, when the microactuator reed is driven by vibrations from the eardrum resulting from sound levels that meet or exceed a damage threshold, control system 126 will exert a force that resists the movement of microactuator reed 350 and, therefore, dampens the motion of the eardrum, protecting it and other elements of the listener's auditory system. Once the sound levels fall below the damage threshold, the magnitude of the vibrations will decrease and control system 126 will allow microactuator reed 350 to vibrate freely.

In embodiments of the invention, drive post 200 may incorporate spring. In embodiments of the invention, drive post 200 may incorporate a shock absorber. In embodiments of the invention, the invention my incorporate a constrained leaf spring, a spring which exerts constant force regardless of displacement. In embodiments of the invention, the constrained leaf spring may be incorporated into drive post 200. In embodiments of the invention, the invention my incorporate a moment arm wherein the resistance changes as a function of displacement. In embodiments of the invention, a moment arm wherein resistance changes as a function of displacement may be incorporated into drive post 200.

In embodiments of the invention, the limiting response may be limited to frequencies where hearing is more susceptible to damage such as, for example, frequencies above 5 KHz.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

REFERENCE NUMBERS

| Number | Element |
|---|---|
| 100 | Contact Hearing Protection Device |
| 101 | Umbrella Structure |
| 102 | Control mechanism |
| 103 | Protective Membrane |
| 104 | Membrane |
| BB | Umbo Region |
| 106 | Magnetic Latching Mechanism |
| 107 | Electromagnet |
| 108 | Control Shaft |
| AA | Annular Region |
| 110 | Travel Stop |
| 111 | Flexible Support |
| 112 | Radial Supports |
| 114 | Membrane Perimeter |
| 114A | Grasping Tab |
| 116 | Connector Ring |
| 118 | Connector Opening |
| 121 | Spring |
| 122 | Non-linear spring |
| 124 | Damper |
| 126 | Control System |
| 128 | Convex Section |
| 129 | Permanent Magnets |
| 130 | Photodetector |
| 130A | Receive Coil |
| 132 | Concave Section |
| 134 | Drive Coil |
| 136 | Electronics Package |
| 140 | Microactuator (Motor) |
| 150 | Sulcus Platform |
| 155 | Perimeter Platform |
| 170 | Chassis |
| 180 | Bias spring (Torsion Spring) |
| 200 | Drive Post |
| 210 | Adhesive (UV) |
| 220 | Umbo Lens |
| 225 | Oil Layer |
| 350 | Microactuator Reed |
| AS | Anterior sulcus |
| BN | Bone |
| CO | Cochlea |
| EC | Ear canal |
| IN | Incus |
| ML | Malleus |
| OS | Ossicles |
| OW | Oval Window |
| SK | Skin |
| ST | Stapes |
| TA | Tympanic Annulus |
| TM | eardrum (eardrum) |
| UM | Umbo |

1. A contact hearing protection device comprising:
a perimeter platform;
a chassis connected to the perimeter platform;
a microactuator connected to the perimeter platform through one or more bias springs at a proximal end thereof, wherein the microactuator comprises a microactuator reed extending from a distal end of the microactuator, the microactuator reed being adapted to move the umbo platform in response to external sound;
a control system including at least one variable limiting element, the control system being connected to the microactuator reed wherein the control element comprises at least one damper connected in parallel with at least one spring element; and
the umbo platform being connected to the microactuator reed at a distal end of the microactuator reed and to the at least one limiting element at a point along the length of the microactuator reed the limiting element increasing the limiting in response to an increased magnitude of movement of the umbo platform.
